(19) United States
(12) Patent Application Publication
(10) Pub. No.: US 2022/0233342 A1
(43) Pub. Date: Jul. 28, 2022

(54) UPPER AIRWAY SUPPORT DEVICE AND METHODS OF USE

(71) Applicant: Shawn Pope, Paris (FR)

(72) Inventor: Shawn Pope, Paris (FR)

(21) Appl. No.: 17/158,760

(22) Filed: Jan. 26, 2021

(51) Int. Cl. A61F 5/56

(52) U.S. Cl. CPC A61F 5/566

(57) ABSTRACT

An intra-oral device extending between a proximal end closer to a patient's mouth and a distal end closer to a bottom of the upper airway, includes a base member configured and arranged to be secured to at least one tooth, a crossbrace coupled to the base member, and a conduit coupled to at least one of the base member and the crossbrace, the conduit spanning the base member and extending to a distance equal to or past an end of the base member adjacent the proximal end.

### FIELD OF THE DISCLOSURE

The present disclosure relates to medical devices for upper airway support. More particularly, the present disclosure relates to devices and methods to provide support for the upper airway while sleeping.

### BACKGROUND OF THE DISCLOSURE

Many sleep disorders are characterized by repetitive episodes of reduced breathing while sleeping. Obstructive Sleep Apnea (OSA), for example, results in episodes of intermittent cessation of airflow while breathing. These interruptions in airflow occur when the tongue and other soft tissue of the nasopharynx and oropharynx either relax during sleep and/or are affected by the flow of air through the upper airway, such as the low pressure generated by the respiratory effort of the patient. In patients with OSA, this may result in a partial or complete blockage of the flow of air through the upper airway. These blockages may lead to a drop in blood oxygen supply, which may cause the patient to enter a lighter stage of sleep or full wakefulness, after which normal respiration resumes. Typically, once the patient falls back asleep, the process of airway blockages and sleep disruptions repeats. Such repetition often continues periodically throughout sleep, in some cases averaging 60 or more episodes per hour of sleep, having consequentially devastating effects on the health and well-being of the patient.

Compressors and facial masks are often used to keep the airway patent during sleep by providing continuous positive airway pressure (CPAP). Such techniques are typically implemented with devices known as CPAP machines. Although CPAP machines are highly effective in treating sleep apnea when they are used correctly and habitually, they have many downsides. The patient must wear a CPAP mask during sleep, which connects to the compressor through a hose, both of which limit mobility and make some sleep positions uncomfortable. CPAP machines do not allow for “natural” breathing, often forcing the patient to breath against positive pressure. CPAP machines often have considerable size and weight and thus may generate some inconveniences in transport. CPAP machines tend to require an external source of electricity and thus are not convenient for those who engage in many outdoor activities (e.g., camping). CPAP machines tend to make sounds during operation, which can disrupt the sleep of the patient and/or sleep partners. CPAP machines have numerous complex electrical parts and require supplies that must be changed on a regular basis. Due to such issues and inconveniences, patient compliance with CPAP therapy may be low, with many patients stopping treatment after a few days, weeks, or months.

Alternative techniques rely on devices that are surgically implanted in tissue in the mouth of the patient to reduce the effects that cause apnea episodes. However, these techniques are far from being commercial and may pose hygienic liabilities. Non-surgical techniques rely on inserting mouth pieces which move the lower jaw forward (e.g., Mandibular Advancement Devices), thus opening an airway behind the tongue. These devices do not treat all types of sleep apnea, however, such as cases where the source of the apnea originates in the nasal cavity. These devices tend to keep the lower jaw in an advanced position by connecting it with the teeth of the upper jaw, which applies force to those teeth, often resulting in tooth pain that becomes evident upon waking. The devices also may cause considerable discomfort by stretching the muscles around the temporomandibular joint, which occurs when the lower jaw is held for a proacted period in an advanced position.

### SUMMARY OF THE DISCLOSURE

In some embodiments, an intra-oral device extending between a proximal end adjacent a patient's mouth and a distal end closer to the upper airway, includes a base member configured and arranged to be secured to at least one tooth, a crossbrace coupled to the base member, and a conduit coupled to at least one of the base member and the crossbrace, the conduit spanning the base member and extending to a distance equal to or past an end of the base member adjacent the proximal end.

### BRIEF DESCRIPTION OF THE DISCLOSURE

Various embodiments of the presently disclosed intra-oral devices are shown herein with reference to the drawings, wherein:

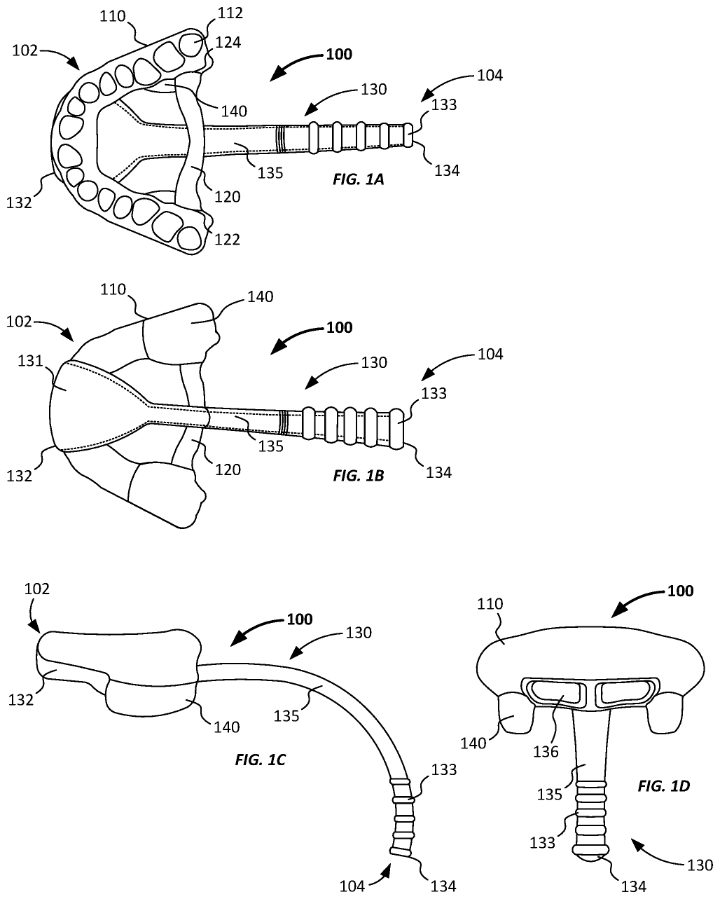

FIGS. 1A-D are schematic top, bottom, side and front views of an intra-oral device for upper airway support;

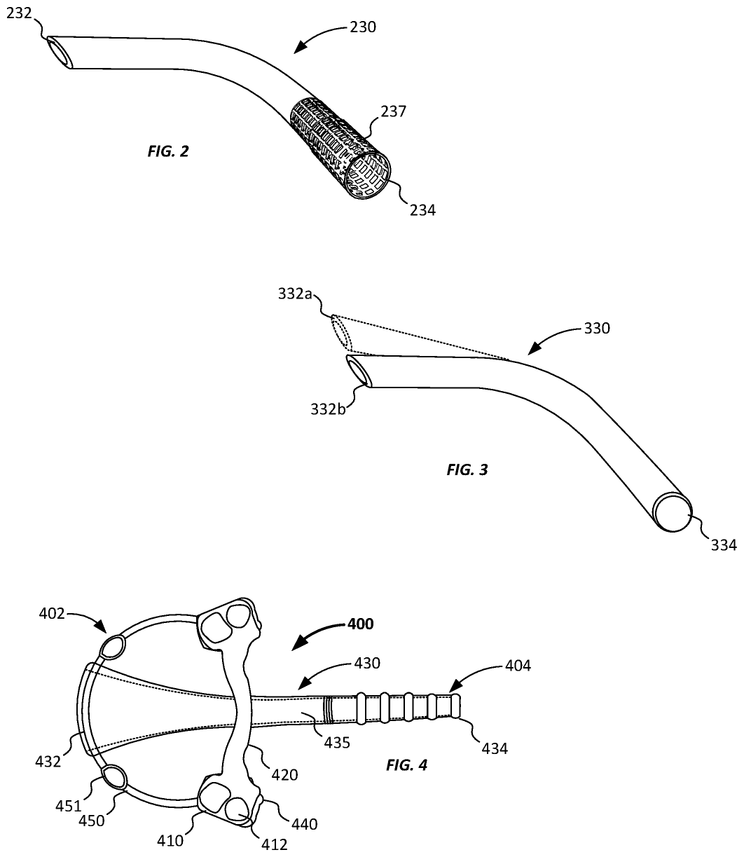

FIG. 2 is a schematic perspective view of one example of a conduit;

FIG. 3 is a schematic perspective view of another example of a conduit; and

FIG. 4 is a schematic top view of another example of an intra-oral device for upper airway support.

Various embodiments of the present invention will now be described with reference to the appended drawings. It is to be appreciated that these drawings depict only some embodiments of the invention and are therefore not to be considered limiting of its scope.

### DETAILED DESCRIPTION

Despite the various improvements that have been made to intra-oral airway support devices and their methods of use, conventional devices suffer from some shortcomings as described above.

There therefore is a need for further improvements to the devices, systems, and methods of providing support for a patient's airway as a sleep aid. Among other advantages, the present disclosure may address one or more of these needs.

As used herein, the term “upper airway” generally refers to the section of the respiratory tract that includes sections of the pharynx, such as the oropharynx and nasopharynx, and may also include the nose and nasal passages, paranasal sinuses, and portions of the larynx above the vocal folds.

Referring now to FIGS. 1A-1D of the drawings, an embodiment of an intra-oral device 100 is shown. The intra-oral device 100 extends between a proximal end 102 and a distal end 104. Intra-oral device 100 generally includes a base member 110 for securing to one or more teeth of the user, a crossbrace 120 connected to the base member 110, and a conduit 130 connected to the base member 110. Although the intra-oral device 100 is depicted as securing to the teeth in the upper jaw of the user, it should be understood to one of ordinary skill in the art that the intra-oral device 100 may be similarly desired and manufactured to secure to the teeth in the lower jaw of the user.

In one example, the base member 110 is formed from an acrylic-based arcuate mold of the teeth of the user. In some examples, base member 110 is manufactured from a hard plastic, but may also be manufactured from any other suitable material for intra-oral use, including, but not limited to, polymers, metals, alloys and the like. In some examples, the base member 110 is manufactured to match elements of the mouth of the user in both size and shape, and includes individual cavities 112 that include impressions which complement the shape of the user's teeth. As such, the base member 110 can be easily secured to, and removed from, the mouth of the user.

In some examples, base member 110 may be custom fitted to the users' teeth in a dental lab, doctor's office, or at home using a boil-and-bite system. Base member 110 may comprise acrylic, wire, polypropylene and similar materials or suitable combinations thereof. Additionally, the base member 110 does not need to be a traditional mouthguard that covers all the teeth. Rather, the base member 110 may be minimized in size (e.g., attaching to the back teeth only, the front teeth only, or any suitable combination). In some examples, a teeth impression is not used and the base material is secured to the teeth using attachment points that secure to structures within the oral cavity (e.g., with wires, magnets, or other suitable techniques). The base member 110 may provide a fixed anchor near the front teeth for the conduit to exit. Additionally, or alternatively, base member 110 may provide an anchor in the middle/back of the mouth for the crossbrace to raise the conduit away from the tongue.

Crossbrace 120 includes two ends, a first end 122 and a second end 124, and may extend laterally, from left to right, across the base member 110. In at least some examples, base member 110 and the crossbrace 120 may be unitarily formed from a single body, or may be formed from separate bodies and securely connected by any suitable technique, including, but not limited to, adhesive or epoxy bonding, injection molding or other suitable technique. In at least some examples, crossbrace 120 is biocompatible. In some examples, crossbrace 120 may be formed of a same material as base member 110. In at least some examples, crossbrace 120 may be coupled to at least one of the base member 110, the conduit 130 and the spacers, and may be used to elevate conduit 130 and prevent it from laying against the tongue. It has been observed that contact between the conduit 130 and the tongue may elicit salivation, which is uncomfortable and diminishes the patient's ability to fall and stay asleep. Thus, by attaching the conduit 130 to crossbrace 120, the conduit may be elevated away from the tongue. Crossbrace 120 may also increase the width of the device, making it impossible or unlikely for the user to swallow it.

Coupled to at least one of base member 110 and crossbrace 120 is a conduit 130 that extends from the proximal end 102 to the distal end 104. Specifically, conduit 130 extends between an inlet 132 and an outlet 134 and defines a lumen 135 that extends between the inlet 132 and the outlet 134. In at least some examples, inlet 132 includes a funnel-shaped member 131 disposed adjacent the proximal end 102, the funnel-shaped member 131 including two separate passages 136 that are positioned and arranged to be disposed between the upper and lower teeth of the patient or user, and to allow air to pass therethrough from the environment into the patient's mouth when the device is being used (See FIG. 1D). Alternatively, a single passage may be formed at inlet 132. Conduit 130 may be formed of a flexible biocompatible tubing that is curved so that the outlet 134 can extend down at least partially through the upper airway. In some examples, conduit 130 may be formed of a semi-flexible/semi-rigid material that is rigid enough to be guided down the throat without collapsing, but flexible enough to follow the curvature of the throat as it is being inserted. The conduit 130 may include one or more circumferential rigid or semi-rigid ribs 133 configured to prevent the conduit from collapsing on itself when disposed within an airway. Alternatively, conduit 130 itself is formed of a material that is rigid enough to prevent this collapse without ribs 133. In some examples, the diameter of the conduit 130 is variable from end-to-end based on the anatomy of the user. In some examples, portions of the conduit 130 (e.g., the circumference, outlet 134, etc.) do not have any sharp edges, so that they do not irritate the sides of the throat. The ends might be wider than the middle of the conduit (e.g., the ends may be funnel shaped or in the form of a small basket). In some examples, conduit 230 may be designed to allow maximal airflow in all directions so that the user can, for example, breathe through the mouth and nose at the same time. Turning to FIG. 2, portions of conduit 230 may have holes in it or be wiffled, perforated, or be in the form of a spiral or mesh (see mesh 237 adjacent outlet end 234 FIG. 2). In this example, the mesh 237 is formed adjacent outlet end 234, but not inlet end 232, but it will be understood that more or less of the conduit 230 may include the mesh as desired. Conduit 130 could also be in the form of a self-expanding stent and therefore made of metallic wire-like material (e.g., stainless steel, nitinol, etc.). The wiffles/holes/perforations of the conduit may be as large as possible without compromising the strength of the conduit, so that even if the device is swallowed, this would not cause asphyxiation.

In some examples, conduit 130 may have various lengths so that it works with users with all oral anatomies (e.g., distances between front teeth and the back of the throat). To avoid reaching so far into the throat as to come into contact with the vocal folds and their immediately surrounding structures, the device 100 may need to be fitted by a professional (e.g., dentist or otolaryngologist) using professional techniques and technologies (e.g., an X-Ray to measure the distance from the front teeth to the hyoid bone or other landmarks in the upper airway). In some examples, conduit 130 may be circular or oblate adjacent distal end 104 (i.e., the part that goes down the throat) and be relatively flatter in the portion adjacent proximal end 102 in the mouth as shown in FIG. 3. This flattening at the proximal end may lessen the profile of the tube, so that it does not crowd the oral cavity. Additionally, conduit 330 may include multiple branches, such as a pair of branches 332a,332b at the inlet that are joined together (FIG. 3). In some examples, at the entrance outside the mouth, the conduit 130 may extend far enough to get past the upper lip. In other words, it may protrude a predetermined distance (e.g., ½ centimeter or more) to prevent the upper lip from blocking the inlet 132. In some examples, the angle of the conduit 130 may be selected so that it is not too steep so as to press down against the tongue, and not too gradual so as to push the soft palate up. In some examples, this angle is customizable according to the user's individual anatomy.

Optionally, a pair of spacers 140 may be coupled to the lower portion of base member 110 and configured to create a gap between the upper teeth and the lower teeth. This gap between the upper and lower teeth may allow the inlet 132 of conduit 130 to sit between the teeth to create a passageway between the external environment and the user's airway. In at least some examples, spacers 140 are formed of a biocompatible material and reside at the bottom of the teeth impression of base member 110 in the back of the mouth or in the middle of the mouth. In at least some examples, the spacers 140 are unitarily formed with base member 110 or formed of the same material as the base member 110. In addition to lowering the jaw to create the gap between the teeth, spacers 140 may serve to create space for the conduit in the oral cavity. Spacers 140 may also prevent users from teeth grinding (bruxism), which could damage the conduit or other portions of the device. Spacers 140 may also provide extra space for the soft palate to fall and prevent the conduit from pushing against the back wall of the throat, which may result in a suffocating feeling. In the mouth, spacers 140 may also lower the lower lip away from the top lip, preventing them from covering the inlet 132.

In one variation, shown in FIG. 4, device 400 includes a base member having at least one cavity 412, a crossbrace 420 spanning the base member 410, and a conduit 430 similar to that previously described with an inlet 432, an outlet 434 and a lumen 435. The device 400 may also include spacers 440 coupled to, or unitarily formed with, base member 410. In this example, base member 410 is much smaller, and configured to be secured to one or two teeth on each side of the mouth. In at least some examples, the base member 410 is configured to be secured to the first and/or second molars only and may include a curved metallic wire (e.g., stainless steel) or plastic hoop 450 to support the conduit 430 at proximal end 402, the hoop 450 being generally flat. Optionally, the hoop 450 may include one or more front attachment points 451 for coupling to central incisors, lateral incisors, and/or cuspids and may be shaped and arranged in a manner suitable for attaching to such teeth.

In use, the patient may place the devices 100 described herein in their mouth prior to sleeping. With the device 100 secured within the mouth via base member 110, the conduit 130 may provide a passage through which oxygen from the environment can enter the patient's mouth and be delivered to the upper airway, the conduit further providing a structural support that prevents the collapse of anatomical features in the upper airway. Crossbrace and spacers may provide the appropriate geometry to protect the device while increasing comfort and performance of the device.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

It will be appreciated that the various dependent claims and the features set forth therein can be combined in different ways than presented in the initial claims. It will also be appreciated that the features described in connection with individual embodiments may be shared with others of the described embodiments.

What is claimed is:

1. An intra-oral device extending between a proximal end configured to be disposed adjacent a patient's mouth and a distal end configured to be disposed closer to a bottom of the upper airway, comprising:
a base member configured and arranged to be secured to at least one tooth;
a crossbrace coupled to the base member; and
a conduit coupled to at least one of the base member and the crossbrace, the conduit spanning the base member and extending to a distance equal to or past an end of the base member adjacent the proximal end.

2. The device of claim 1, further comprising at least one spacer coupled to the base member.

3. The device of claim 2, wherein the at least one spacer is unitarily formed with the base member.

4. The device of claim 2, wherein the at least one spacer comprises a pair of spacers coupled to a bottom of the base member.

5. The device of claim 1, wherein the crossbrace is configured and arranged to elevate the conduit away from the patient's tongue.

6. The device of claim 1, wherein the base member includes at least one cavity that includes an impression of a tooth of the patient.

7. The device of claim 6, wherein the base member includes a plurality of cavities for securing to upper teeth of the patient.

8. The device of claim 1, wherein the conduit includes at least one inlet and an outlet.

9. The device of claim 8, wherein the conduit includes multiple inlet branches that join together at a common lumen.

10. The device of claim 8, wherein the at least one inlet has a flattened shape.

11. The device of claim 1, wherein the conduit includes a portion that has a mesh, wiffled, or perforated structure.

12. The device of claim 1, wherein the conduit extends past the base member adjacent the proximal end.

13. A method of supporting an upper airway, comprising:
providing an intra-oral device that extends between a proximal end closer to a patient's mouth and a distal end closer to the upper airway, the intra-oral device including a base member configured and arranged to be secured to at least one tooth, a crossbrace coupled to the base member, and a conduit coupled to at least one of the base member and the crossbrace, the conduit spanning the base member and extending to a distance equal to or past an end of the base member adjacent the proximal end; and
placing the intra-oral device in the patient's mouth so that the base member is disposed in the oral cavity and at least a portion of the conduit is disposed in the upper airway.

14. The method of claim 13, further comprising the step of providing at least one spacer coupled to the base member and configured and arranged to provide a gap between the upper teeth and lower teeth.

15. The method of claim 13, further comprising the step of elevating the conduit away from the patient's tongue with the crossbrace.

16. The method of claim 13, further comprising the step of taking an impression of at least one tooth of the patient and modifying the base member so that it matches the impression.

17. The method of claim 16, further comprising the step of securing the base member to upper teeth of the patient.

18. The method of claim 13, wherein placing the intra-oral device in the patient's mouth comprises extending the conduit out of the patient's mouth so that an inlet of the conduit extends past teeth of the patient.
